(19) United States
(12) Patent Application Publication
(10) Pub. No.: US 2003/0082235 A1
(43) Pub. Date: May 1, 2003

(54) Novel reverse thermo-sensitive block copolymers

(75) Inventors: Daniel Cohn, Jerusalem (IL); Alejandro Sosnik, Jerusalem (IL); Michael Kheyfetz, Jerusalem (IL)

Correspondence Address: FOLEY AND LARDNER, SUITE 500, 3000 K STREET NW, WASHINGTON, DC, 20007, US

(73) Assignee: Yissum Research Development Company of the Hebrew University of Jerusalem

(21) Appl. No.: 10/211,228

(22) Filed: Aug. 5, 2002

Related U.S. Application Data
(60) Provisional application No. 60/311,382, filed on Aug. 13, 2001.

(51) Int. Cl. A61K 9/14

(52) U.S. Cl. 424/486

(57) ABSTRACT

The invention provides a responsive polymeric system, comprising: a polymeric responsive component capable of undergoing a transition that results in a sharp increase in viscosity in response to a change in temperature at a predetermined body site; wherein the polymeric component comprises hydrophilic and hydrophobic segments covalently bound within the polymer component, by at least one chain extender or coupling agent, having at least 2 functional groups; wherein the hydrophilic and hydrophobic segments do not display Reverse Thermal Gelation behavior of their own at clinically relevant temperatures and; wherein the viscosity of the polymeric component increases by at least about 2 times upon exposure to a predetermined trigger.

This application claims priority from provisional U.S. application, serial No. 60/311382, filed Aug. 13^{th}, 2001, and incorporated herein by reference in its entirety.

The present invention relates to novel reverse thermo-responsive polymeric systems More specifically, the present invention relates to a polymeric system comprising an environmentally responsive polymeric component based on the chemical binding of hydrophobic and hydrophillic segments combined in alternating or random chain order, which is introducible into the body in aqueous solution and which undergoes a substantial change in viscosity at a predetermined body site, said polymeric system being useful in drug delivery systems, in the prevention of post-surgical adhesions, as a sealant, in tissue engineering and in numerous other biomedical applications.

### BACKGROUND OF THE INVENTION

There is a wide variety of materials which are foreign to the human body and which are used in direct contact with its organs, tissues and fluids. These materials are called Biomaterials, and they include, among others, polymers, ceramics, biological materials, metals, composite materials and combinations thereof.

The development of polymers suitable to be implanted without requiring a surgical procedure, usually named injectable polymers, has triggered much attention in recent years. These materials combine low viscosity at the injection stage, with a gel or solid consistency developed in situ, later on. The systems of the present invention are preferably used, without limitation, as matrices for the controlled release of biologically active agents, as sealants, as coatings and as barriers in the body. The area of Tissue Engineering represents an additional important field of application of the improved responsive systems disclosed hereby, where they can perform as the matrix for cell growth and tissue scaffolding.

The syringability of injectable biomedical systems is their most essential advantage, since it allows their introduction into the body using minimally invasive techniques. Furthermore, their low viscosity and substantial flowability at the insertion time, enable them to reach and fill spaces, otherwise unaccessible, as well as to achieve enhanced attachment and improved conformability to the tissues at the implantation site. On the other hand, a sharp increase in viscosity is a fundamental requirement for these materials to be able to fulfill any physical or mechanical function, such as sealing or performing as a barrier between tissue planes. The high viscosities attained also play a critical role in generating syringable materials that, once at the implantation site, are also able to control the rate of release of drugs or can function as the matrix for cell growth and tissue scaffolding. Clearly, biodegradability is yet another important requirement for some of these materials.

A polymer network is characterized by the positive molecular interactions existing between the different components of the system. These intereractions may be physical in nature, such as chain entanglements, or chemical such as ionic interactions, hydrogen bonding, Van der Waals attractions and covalent bonding. Bromberg et al. (U.S. Pat. No. 5,939,485 ) developed responsive polymer networks exhibiting the property of reversible gelation triggered by a change in diverse environmental stimuli, such as temperature, pH and ionic strength. Pathak et al. (U.S. Pat. No. 6,201,065) disclosed thermo-responsive macromers based on cross-linkable polyols, such as PEO-PPO-PEO triblocks, capable of gelling in an aqueous solution. The macromers can be covalently crosslinked to form a gel on a tissue surface in vivo. The gels are useful in a variety of medical applications including drug delivery.

The term “thermo-sensitive” refers to the capability of a polymeric system to achieve significant chemical, mechanical or physical changes due to small temperature differentials. The resulting change is based on different mechanisms such as ionization and entropy gain due to water molecules release, among others (Alexandridis and Hatton, Colloids and Surfaces A, 96, 1 (1995)). Since one of their fundamental advantages is to avoid the need for an open surgical procedure, thermo-responsive materials are required to be easily syringable, combining low viscosity at the injection stage, with a gel or solid consistency being developed later on, in situ.

Thermosensitive gels can be classified into two categories: (a) if they have an upper critical solution temperature (UCST), they are named positive-sensitive hydrogels and they contract upon cooling below the UCST, or (b) if they have a lower critical solution temperature (LCST), the are called negative-sensitive hydrogels and they contract upon heating above this temperature.

The reverse thermo-responsive phenomenon is usually known as Reverse Thermal Gelation (RTG) and it constitutes one of the most promising strategies for the development of injectable systems. The water solutions of these materials display low viscosity at ambient temperature, and exhibit a sharp viscosity increase as temperature rises within a very narrow temperature interval, producing a semi-solid gel once they reach body temperature. There are several RTG displaying polymers. Among them, poly(N-isopropyl acrylamide) (PNIPAAm) (Tanaka and co-workers in U.S. Pat. No. 5,403,893 and Hoffman A. S. et al., J. Controlled Release, 6, 297 (1987)). Unfortunately, poly(N-isopropyl acrylamide) is non-degradable and, in consequence, is not suitable for a diversity of applications where biodegradability is required. Additionally, the N-isopropylacrylamide is toxic.

One of the most important RTG-displaying materials is the family of poly(ethylene oxide)/poly(propylene oxide)/poly(ethylene oxide) (PEO-PPO-PEO) triblocks, available commercially as Pluronic™ (Krezanoski in U.S. Pat. No. 4,188,373). Adjusting the concentration of the polymer, renders the solution with the desired liquid-gel transition. However, relatively high concentrations of the triblock (typically above 15-20%) are required to produce compositions that exhibit such a transition or even a minor transition, at commercially or physiologically useful temperatures. Another known system which is liquid at room temperature, and becomes a semi-solid when warmed to about body temperature, is disclosed in U.S. Pat. No. 5,252,318, and consists of tetrafunctional block polymers of polyoxyethylene and polyoxypropylene condensed with ethylenediamine (commercially available as Tetronic.™

The endothermic phase transition taking place, is driven by the entropy gain caused by the release of water molecules bound to the hydrophobic groups in the polymer backbone. Unfortunately, despite their potential, some fundamental aspects of their performance severely restrict their clinical use. Even though these materials exhibit a significant increase in viscosity when heated up to 37° C., the levels of viscosity attained are not high enough for most clinical applications. Derived from this fundamental limitation, these systems display unsatisfactory mechanical properties and unacceptably short residence times at the implantation site. Furthermore, due to these characteristics, these gels have high permeabilities, a property which renders them unsuitable for drug delivery applications because of the fast drug release kinetics of these gels. Despite their clinical potential, these materials have failed to be used successfully in the clinic, because of serious performance limitations (Steinleitner et al., Obstetrics and Gynecology, 77, 48 (1991) and Esposito et al., Int. J. Pharm. 142, 9 (1996)).

Cohn et al. (U.S. Patent Application 60/138,132) disclosed high molecular weight PEO-PPO-PEO polymers obtained by the polymerization of the native Pluronic.™ triblocks with different chain extenders. An important drawback of these relatively high molecular weight polymers pertain to the non-biodegradability of the basic triblock and often also of the polymers themselves, being, therefore, removed with difficulty through the kidneys (Jeong et al., Nature, 388, 360-2 (1998)). Furthermore, the properties of the polymers disclosed by Cohn et a/ are limited by the existing PEO-PPO-PEO triblocks and therefore, by the PEO/PPO ratio and the molecular weight of these commercially available triblocks. Also, several of these triblocks have proved to be toxic. It should also be stressed that in the case of the polymers displayed in U.S. Patent Application 60/138,132, the two fundamental characteristics defining its rhelogical behavior in the clinic (namely, Tᵢ, the temperature at which the viscosity raises dramatically and the viscosity at 37° C.), are interrrelated and cannot be tailored into the system independently. The ability to design materials, the Ti of which can be programmed into the system, so it is lower that, close to or above T_{OR}, in one hand, and considerably lower that 37° C. or close to it, in the other hand, represents an important clinical advantage. In light of the above, it is apparent that the materials of the present invention have substantial advantages, and overcome important limitations and drawbacks of the materials of the prior art.

Biodegradability plays a unique role in a diversity of devices, implants and prostheses. Their most obvious advantage pertains to the fact that there is no need to remove the system, once it has accomplished its objectives. In addition, they can perform as matrices for the release of bioactive molecules and result in improved healing and tissue regeneration processes. Biodegradable polymers such as polyesters of α-hydroxy acids, like lactic acid or glycolic acid, are used in diverse applications such as bioabsorbable surgical sutures and staples, some orthopedic and dental devices, drug delivery systems and more advanced applications such as the absorbable component of selectively biodegradable vascular grafts, or as the temporary scaffold for tissue engineering. Biodegradable polyanhydrides and polyorthoesters having labile backbone linkages, have been developed, the disclosures of which are incorporated herein. Polymers which degrade into naturally occurring materials, such as polyaminoacids, also have been synthesized. Degradable polymers formed by copolymerization of lactide, glycolide, and ε-caprolactone have been disclosed. Polyester-ethers have been produced by copolymerizing lactide, glycolide or ε-caprolactone with polyethers, such as polyethylene glycol (“PEG”), to increase the hydrophilicity and degradation rate.

Unfortunately, the few absorbable polymers clinically available today are stiff, hydrophobic solids which are, therefore, clearly unsuitable for non-invasive surgical procedures, where injectability is a fundamental requirement. The only way to avoid the surgical procedure with these polymers, is to inject them as micro or nanoparticles or capsules, typically containing a drug to be released. As an example, injectable implants comprising calcium phosphate particles in aqueous viscous polymeric gels, were first proposed by Wallace et al. in U.S. Pat. No. 5,204,382. Even though these the ceramic component is generally considered to be nontoxic, the use of nonabsorbable particulate material seems to trigger a foreign body response both at the site of implantation as well as at remote sites, due to the migration of the particles, over time.

Among the approaches developed, the in situ precipitation technique developed by R. Dunn, as disclosed in U.S. Pat. No. 4,938,763, is one strategy worth mentioning. These systems comprise a water soluble organic solvent, in which the polymer is soluble. Once the system is injected, the organic solvent gradually dissolves in the aqueous biological medium, leaving behind an increasingly concentrated polymer solution, until the polymer precipitates, generating the solid implant in situ. A similar approach has been reported by Kost et al (J. Biomed. Mater. Res., 50, 388-396 (2000)).

In situ polymerization and/or crosslinking is another important technique used to generate injectable polymeric systems. Hubbell et al described in U.S. Pat. No. 5,410,016, water soluble low molecular precursors having at least two polymerizable groups, that are syringed into the site and then polymerized and/or crosslinked in situ chemically or preferably by exposing the system to UV or visible radiation. Mikos et al (Biomaterials, 21, 2405-2412 (2000)) described similar systems, whereas Langer et al (Biomaterials, 21, 259-265 (2000)) developed injectable polymeric systems based on the percutaneous polymerization of precursors, using UV radiation. An additional approach was disclosed by Scopelianos and co-workers in U.S. Pat. No. 5,824,333 based on the injection of hydrophobic bioabsorbable liquid copolymers, suitable for use in soft tissue repair.

Unfortunately, all these techniques have serious drawbacks and limitations, which significantly restrict their applicability. The paradox in this area has to do, therefore, with the large gap existing between the steadily increasing clinical demand for Injectables, on one hand, and the paucity of materials suitable to address that need, on the other hand.

### OBJECTS OF THE INVENTION

It is an object of this invention to provide novel polymeric reverse thermo-responsive compositions, for diverse applications, preferably in the biomedical field, selected from a group consisting of drug delivery systems, the prevention of post-surgical adhesions, sealants and the Tissue Engineering field, among numerous others, designed to cover a broad range of properties. The compositions disclosed hereby can be brought to the implantation site via non-invasive surgical procedures or open surgery, as well as being deployed to the location in any other way. In the case of biodegradable systems, these materials are engineered to display different degradation kinetics. This was achieved by generating novel amphiphillic copolymeric compositions, combining hydrophobic and hydrophillic segments, which allowed to achieve the desired Reverse Thermal Gelation (RTG) behavior.

According to the present invention there is now provided a responsive polymeric system, comprising novel amphiphiles obtained by the combination of both hydrophobic and hydrophillic basic segments, which, separately, do not display any kind of clinically relevant viscosity change of their own, and are capable of undergoing a transition that results in a sharp increase in viscosity in response to a triggering effected at a predetermined body site and an aqueous-based solvent wherein the viscosity of said polymeric component increases by at least about 2 times upon exposure to a predetermined trigger.

More specifically, according to the present invention, there is now provided a responsive polymeric system, comprising a polymeric responsive component capable of undergoing a transition that results in a sharp increase in viscosity in response to a change in temperature at a predetermined body site; wherein the polymeric component comprises hydrophilic and hydrophobic segments covalently bound within said polymer component, by at least one chain extender or coupling agent, having at least 2 functional groups; wherein the hydrophilic and hydrophobic segments do not display Reverse Thermal Gelation behavior of their own at clinically relevant temperatures and; wherein the viscosity of said polymeric component increases by at least about 2 times upon exposure to a predetermined trigger.

In preferred embodiments of the present invention said responsive polymeric component has a formula selected from a group consisting of:

a) [-Xₙ-A-Xₙ-E-B-E-]ₘ

b) [-Xₙ-B-Xₙ-E-A-E-]ₘ,

c) M-Xₙ-E-B-E-Xₙ-M

d) N-Xₙ-E-A-E-Xₙ-N

e) [-E-Xₙ-A(Xₙ)_{y}(E)_{y}(B)_{y}-Xₙ-E-B-]ₘ and

f) [-E-Xₙ-B(Xₙ)_{y}(E)_{y}(A)_{y}-Xₙ-E-A-]ₘ;

wherein segments A are bifunctional, trifunctional or multifunctional hydrophilic segments and M are monofunctional hydrophilic segments, respectively; wherein segments B are bifunctional, trifunctional or multifunctional hydrophobic segments and N are monofunctional hydrophobic segments, respectively; wherein segments X are bifunctional degradable segments; wherein E are bi, tri or multifunctional chain extenders or coupling molecules, wherein n and m denote the respective degrees of polymerization and y designates the additional functionality of the segment above 2.

In a preferred embodiment of the present invention said predetermined trigger is temperature, the system displaying said increase in viscosity when being heated up, preferably from a lower temperature to body temperature and more preferably from room temperature to body temperature.

As stated, the present invention introduces a novel group of polymeric compositions based on the following generic formulae:

a) [-Xₙ-A-Xₙ-E-B-E-]ₘ

b) [-Xₙ-B-Xₙ-E-A-E-]ₘ,

c) M-Xₙ-E-B-E-Xₙ-M

d) N-Xₙ-E-A-E-Xₙ-N

e) [-E-Xₙ-A(Xₙ)_{y}(E)_{y}(B)_{y}-Xₙ-E-B-]ₘ and

f) [-E-Xₙ-B(Xₙ)_{y}(E)_{y}(A)_{y}-Xₙ-E-A-]ₘ;

wherein A is a hydrophilic bifunctional segment selected from a group consisting of —OH, —SH, —COOH, —NH₂, —CN or —NCO group terminated poly(oxoethylene) or any other bifunctional hydrophilic segment having the appropriate terminal group, or a trifunctional segment selected from a group consisting in poly(oxoethylene triol), poly(oxoethylene triamine), poly(oxoethylene triacarboxylic acid), ethoxylated trimethylolpropane, or any other trifunctional hydrophilic segment having the appropriate terminal group, or other multifunctional segment, most preferably bifunctional, and/or combinations thereof.

B is a hydrophobic bifunctional component is selected from a group consisting of a —OH, —SH, —COOH, —NH₂, —CN or —NCO group terminated polyoxyalkylene polymer (selected from a group consisting of poly(propylene glycol) (PPG), polyoxopropylene diamine (Jeffamine.™), polytetramethylene glycol (PTMG)), polyesters selected from a group consisting of poly(caprolactone), poly(lactic acid), poly(glycolic acid) or combinations or copolymers thereof, polyamides or polyanhydrides or any other bifunctional hydrophobic segment having the appropriate terminal group, or a trifunctional segment selected from a group consisting of poly(oxopropylene triol), poly(oxopropylene triamine), poly(oxopropylene triacarboxylic acid), or any other trifunctional hydrophobic segment, having the appropriate terminal group, or other multifunctional hydrophobic segment, most preferably bifunctional segment, and combinations thereof.

E is a chain extender or coupling molecule is bifunctional reactive molecule selected from a group consisting of phosgene, aliphatic or aromatic dicarboxylic acids or any other reactive derivative (selected from a group consisting of oxalyl chloride, malonyl chloride, succinyl chloride, glutaryl chloride, fumaryl chloride, adipoyl chloride, suberoyl chloride, pimeloyl chloride, sebacoyl chloride, terephtaloyl chloride, isophtaloyl chloride, phtaloyl chloride and/or mixtures thereof or other dicarboxylic acid derivative), aminoacids selected from a group consisting of glycine, alanine, valine, phenylalanine, leucine, isoleucine or any other essencial aminoacid or not, oligopeptides selected from a group consisting of RGD, RGD-S or any other oligopeptide having or not biological activity, aliphatic or aromatic diamines selected from a group consisting of ethylene diamine, propylene diamine, butylene diamine, or any other diamine or amine derivative, aliphatic or aromatic diols selected from a group consisting of ethylene diol, propanediol, butylenediol or any other diol, aliphatic or aromatic diisocyanates selected from a group consisting of hexamethylene diisocyanate, methylene bisphenyldiisocyanate, methylene biscyclohexanediisocyanate, tolylene diisocyanate or isophorone diisocyanate or any other bifunctional reactive molecule, having the appropriate terminal group or trifunctional reactive molecules selected from a group consisting of cyanuric chloride, triisocyanates, triamines, triols, aminoacids selected from a group consisting of lysine, serine, threonine, methionine, asparagine, glutamate, glutamine, histidine or any other essencial aminoacid or not having three functional groups, oligopeptides or any other trifunctional reactive molecule, having the appropriate terminal groups or multifunctional couplig molecule, most preferably phosgene, diisocyantes, aminoacids, oligopeptides or bifunctional carboxylic acid derivatives, and combinations thereof. E may also comprise combinations of the functional groups described above in the same molecule. The reaction products are poly(ether-carbonate)s, poly(ether-ester)s, poly(ether-urethane)s or derivatives of chlorotriazine, most preferably poly(ether-carbonate)s, poly(ether-ester)s or poly(ether-urethanes), polyimides, polyureas and combinations thereof.

M is a hydrophilic monofunctional segment, selected from a group consisting of —OH, —SH, —COOH, —NH₂, —CN or —NCO group terminated poly(oxoethylene) monomethylether or any other monofunctional hydrophilic segment, having the appropriate terminal group and combinations thereof.

N is an hydrophobic monofunctional segment, selected from a group consisting of —OH, —SH, —COOH, —NH₂, —CN or —NCO group terminated poly(oxopropylene) monomethylether or any other monofunctional hydrophobic segment, having the appropriate terminal group and combinations thereof.

Segment X renders the molecule degradable due to its hydrolytic instability and is based preferably on segments selected from a group consisting of aliphatic or aromatic ester, amide or anhydride groups formed from α-hydroxy carboxylic acid units or their respective lactones, selected from a group consisting of lactide, glycolide or ε-caprolactone, their respective lactams or the respective poly(anhydride)s. The X segments comprise preferably hydroxy carboxylic units or their respective lactones, or similar compounds selected from a group and without limitation consisting of lactic acid, lactide, ε-caprolactone, glycolic acid, glycolide, β-propiolactone, δ-glutarolactone, δ-valerolactone, β-butyrolactone, ethylene carbonate, trimethylene carbonate, γ-pivalactone, α,α-diethylpropiolactone, p-dioxanone, 1,4-dioxepan-2-one, 3-methyl-1,4 dioxanone-2,5-dione, 3,3-dimethyl-1,4-dioxanone-2,5-dione, cyclic esters of α-hydroxybutyric acid, α-hydroxyvaleric acid, α-hydroxyisovaleric acid, α-hydroxycaproic acid, α-hydroxy-α-ethylbutyric acid, α-hydroxyisocaproic acid, α-hydroxy-α-methylvaleric acid, α-hydroxypentanoic acid, α-hydroxystearic acid, α-hydroxylignoceric acid, salycilic acid and mixtures, thererof or amino carboxylic units, such as caprolactam, laurolactam, lactamide and mixtures, thereof.

In the present invention n and m denote the respective degrees of polymerization and y represents the additional functionality above 2. Thus, the total functionality of the segment will be y+2. For example, when a trifunctional A segment is present in the compositions disclosed hereby, y will be equal to 1.

Aqueous solutions of the polymers of this invention display from slight to remarkable reverse thermal gelation (RTG) characteristics: they combine the properties of low viscosity liquids at low temperatures (preferably around RT), with intermediate to high viscosities at higher temperatures at body temperature.

The novel, tailor-made compositions of the present invention display beneficial properties unattainable by the prior art by capitalizing, in a unique and advantageous way, on the Reverse Thermal Gelation phenomenon displayed by the fine-tuned combination of hydrophilic and hydrophobic native segments in the adequate and desired balance, in order to achieve the required viscosity change profile.

It is an additional object of the invention to introduce hydrolytically unstable segments along the polymeric backbone, allowing, therefore, to fine tune both the degradation rate of the polymer molecule as well as control the stability of the whole system and its rheological properties. It is an additional object of the invention to render these compositions with specific biological functions by incorporating biomolecules of various types, physically (by blending them into the system) or chemically (by covalently binding them to the polymer). It is an additional object of the invention to incorporate cells of various types into these materials, for them to perform as RTG-displaying matrices for cell growth and tissue scaffolding. It is an additional object of the invention to introduce inorganic components of biological origin.

Preferably said responsive polymeric component is biodegradable.

In especially preferred embodiments of the present invention said hydrophobic monofunctional component is selected from a group consisting of hydroxy, amine, tiol, cyano, isocyanate or carboxylic acid-terminated poly(propylene glycol) monomethylether, poly(tetramethylene glycol) monomethylether, poly(caprolactone) monomethylether, poly(lactic acid) monomethylether or any other monofunctional hydrophobic segment, having the appropriate terminal group, or a bifunctional component is selected from a group consisting of a —OH, —SH, —COOH, —NH₂, —CN or —NCO terminated polyoxyalkylene polymer, polyester, polyamide, polyurethane, polycarbonate or polyanhydride or any other bifunctional hydrophobic segment having the appropriate terminal group, or a trifunctional segment selected from a group consisting of poly(oxopropylene triol), poly(oxopropylene triamine), poly(oxopropylene triacarboxylic acid), or any other trifunctional hydrophobic segment, having the appropriate terminal group, or other multifunctional hydrophobic segment, most preferably bifunctional segment, and combinations thereof, and the hydrophilic monofunctional segment is selected from a group consisting of hydroxy, amine, tiol, cyano, isocyanate or carboxylic poly(ethylene glycol) monomethylether or any other monofunctional hydrophilic segment, having the appropriate terminal group, or bifunctional segment selected from a group consisting of —OH, —SH, —COOH, —NH₂, —CN or —NCO group terminated poly(oxoethylene) or any other bifunctional hydrophilic segment having the appropriate terminal group, or a trifunctional segment selected from a group consisting in poly(ethylene triol), poly(oxoethylene triamine), poly(oxoethylene triacarboxylic acid), ethoxylated trimethylolpropane, or any other trifunctional hydrophilic segment having the appropriate terminal group, or other multifunctional segment, most preferably bifunctional, and combinations thereof.

In further preferred embodiments of the present invention said responsive component is a segmented block copolymer comprising polyethylene oxide (PEO) and polypropylene oxide (PPO) chains, wherein said PEO and PPO chains are connected via a chain extender, wherein said chain extender is a bifunctional, trifunctional or multifunctional molecule selected from a group consisting of phosgene, aliphatic or aromatic dicarboxylic acids, their reactive derivatives such as acyl chlorides and anhydrides, diamines, diols, aminoacids, oligopeptides, polypeptides, or cyanuric chloride or any other bifunctional, trifunctional or multifunctional coupling agent, or other molecules, synthetic or of biological origin, able to react with the mono, bi, tri or multifunctional —OH, —SH, —COOH, —NH₂, —CN or —NCO group terminated hydrophobic and hydrophilic components or any other bifunctional or multifunctional segment, and/or combinations thereof.

As indicated hereinbefore, preferrably said responsive component contains molecule/s, to be delivered into the body.

Preferrably said responsive component contains living cells or other materials of tissular origin.

Compositions according to this invention are suitable to be used in the human body, preferably in applications where the combination of ease of insertion and enhanced initial flowability, on one hand, and post-implantation high viscosity and superior mechanical properties, on the other hand, are required.

Aiming to expand the clinical applicability of the RTG polymers, it is an object of this invention to provide enhanced reverse thermo-responsive polymers. These materials will find a variety of important applications, and without limitation, in the biomedical field, such as in non-invasive surgical procedures, as matrices for the controlled release of biologically active agents (drug delivery systems), as sealants, as coatings and lubricants, as transient barriers in the body aiming at reducing or preventing of adhesions subsequent to surgical procedures and in the Tissue Engineering field where they can perform as the matrix for cell growth and tissue scaffolding. The different polymeric compositions may be non-biodegradable or biodegradable, depending on their composition, as dictated by the application in which the composition is to be used and they are engineered to display different degradation kinetics, designed to cover a broad range of mechanical properties. This was achieved by combining various biodegradable segments along the polymeric backbone that display diverse degradation kinetics and diverse functional groups having different sensitivity to hydrolysis.

The novel compositions of the present invention are tailor-made, by capitalizing on the uniqueness of the Reverse Thermal Gelation phenomenon. The endothermic phase transition taking place, is driven by the entropy gained due to the release of water molecules bound to the hydrophobic groups in the polymer backbone. Its clear, therefore, that, in addition to molecular weight considerations and chain mobility parameters, the balance between hydrophilic and hydrophobic moieties in the molecule, plays a crucial role. Consequently, the properties of different materials were adjusted and balanced by variations of the basic chemistry, composition and molecular weight of the different components.

To illustrate the scope of the work conducted, suffice to mention the new, minimally invasive approaches for intracardiac surgery, as well as the novel injectable materials investigated for use in various areas such as Tissue Engineering, the treatment of craniofacial arteriovenous defects, and bone surgery.

The term ‘viscosity’ is used to describe the fundamental characteristic of the water solutions generated by the polymeric compositions disclosed hereby, which related to the resistance of the composition to flow. For purposes of the present invention, viscosity is measured in centiPoise (cP) units or Pa.s, where 1000 cP=10 Poise=1 Pa.s, as determined by a Brookfield Programmable Viscometer using the required DV-II+spindle at 0.05 rpm.

In the invention disclosed herein, the chain extension or crosslinking of low molecular weight precursors or the coupling of monofunctional blocks, is performed using a variety of bifunctional, trifunctional or multifunctional molecules, preferably phosgene, diacyl chlorides or their reactive derivatives, cyanuric chloride, aminoacids or oligopeptides, most preferably phosgene or acyl derivatives. The reaction products contain, therefore, carbonate moieties or derivatives of chlorotriazine, among others, most preferably carbonates or diurethanes. The polymers of the present invention can also have additional structures, such as grafted systems.

The materials described in this invention are generated following more then one synthetic scheme. For example, a one-step process, wherein the hydrophilic and the hydrophobic segments are phosgenated, the hydrophilic segment being selected from a group consisting of poly(ethylene glycol) or any other derivative (or the respective biodegradable triblocks), obtained separately, react with relatively hydrophobic chain selected from a group consisting of poly(propylene glycol) (PPG), poly(tetramethylene glycol) (PTMG), polycaprolactone, polylactic acid, polyglycolic acid or any other hydrophobic chain in a second condensation reaction or the opposite, phosgenated poly(propylene glycol) or poly(tetramethylene glycol) (PTMG) segments or any other derivate or hydrophobic chain (or the respective biodegradable triblocks), obtained separately, react with relatively hydrophylic chains, selected from a group consisting of poly(ethylene glycol) (PEG) or any other derivative or other hydrophilic block.

### SYNTHESIS OF POLYMERS ACCORDING THE PRESENT INVENTION

a) Alternating polymers (-[A-B]-)

In order to synthesize the Xₙ-A-Xₙ, Xₙ-B-Xₙ, M-Xₙor N-Xn tri or diblocks, the hydroxy, amine or carboxylic acid-terminated hydrophilic bifunctional segment A selected from a group consisting of poly(ethylene oxide), or the hydroxy, amine or carboxylic acid-terminated hydrophobic segment B selected from a group consisting of poly(propylene oxide), or the hydroxy, amine or carboxylic acid-terminated monofunctional hydrophilic segments M selected from a group consisting of poly(ethylene oxide) monomethyl ether or the polyoxoalkylene monoamine, or the hydroxy, amine or carboxylic acid-terminated monofunctional hydrophobic segments N, are reacted with the hydroxyacid, the respective lactone, the respective lactam or a related monomer as otherwise described herein, to produce an Xₙ-A-Xₙ or Xₙ-B-Xn triblock or an M-Xₙ or N-Xₙ diblock. Once the triblock or diblock is formed, it is reacted with the chain extender E at certain conditions in order to produce the pentablock of structure E-Xₙ-A-Xₙ-E or E-Xₙ-B-Xₙ-E and triblock M-Xₙ-E or N-Xₙ-E, respectively. Then, the pentablock E-Xₙ-A-Xₙ-E or triblock M-Xₙ-E is reacted with the hydrophobic segment B in order to obtain the polymer [-E-Xₙ-A-Xₙ-E-B-]ₘ or M-Xₙ-E-B-E-Xₙ-M, and the pentablock E-Xₙ-B-Xₙ-E or triblock N-Xₙ-E is reacted with the hydrophilic segment A in order to obtain the polymer [-E-Xₙ-B-Xₙ-E-A-]ₘ or N-Xₙ-E-A-E-Xₙ-N, respectively. The synthesis of polymers with n=0, is carried out eliminating the first step of formation of the Xₙ-A-Xₙ or Xₙ-B-Xₙ triblocks or M-Xₙ or N-Xₙ diblocks, and the bifunctional segment A or B or monofunctional segment M is reacted directly with the chain extender E.

When a higher functionaly is desired in A or B, the first step will render tetra or multiblocks. In these cases the general formula of the first step products is: Xₙ-A(Xₙ)ₓ-Xₙ or Xₙ-B(Xₙ)_{y}-Xₙ, when y denotes the additional Xₙ segments connected to the multifunctional segment. This can be illustrated by the case where trifunctional A or B segments (y=1) are present, the general formula being then: Xₙ-A(Xₙ)-Xₙ or Xₙ-B(Xₙ)-Xₙ. In the case of tetrafunctional blocks (y=2), the general formula will be: Xₙ-A(Xₙ)₂-Xₙ Xₙ or Xₙ-B(Xₙ)₂-Xₙ Once the tetra or multiblocks are formed, they are reacted with the chain extender E at certain conditions in order to produce the multiblocks of structure E-Xₙ-A(Xₙ)_{y}(E)_{y}-Xₙ-E or E-Xₙ-B(Xₙ)_{y}(E)_{y}-Xₙ-E. Then, the multiblock E-Xₙ-A(Xₙ)_{y}(E)_{y}-Xₙ-E is reacted with the hydrophobic segment B in order to obtain the polymer [-E-Xₙ-A(Xₙ)_{y}(E)_{y}(B)_{y}-Xₙ-E-B-]ₘ, and the multiblock E-Xₙ-B(Xₙ)_{y}(E)_{y}-Xₙ-E is reacted with the hydrophilic segment A in order to obtain the polymer [-E-Xₙ-B(Xₙ)_{y}(E)_{y}(A)_{y}-Xₙ-E-A-]ₘ, respectively.

A particularly preferred synthesis of the triblock Xₙ-A-Xₙ or Xₙ-B-Xₙ or diblock M-Xₙ or N-Xₙ according to the present invention, relies on the use of the cyclic ester or amide of the hydroxyacid selected from a group consisting of, and without limitation, lactic acid, glycolic acid, caprolactone, lactamide, caprolactam or any other reactive derivate.

The synthesis of the triblock Xₙ-A-Xₙ or Xₙ-B-Xₙ or the diblock M-Xₙ or N-Xₙ or multiblock Xₙ-A(Xₙ)ₓ-Xₙ or Xₙ-B(Xₙ)_{y}-Xₙ preferably proceeds by way of a ring-opening mechanism, whereby the opening of the lactones, lactams or anhydrides, selected from a group consisting of caprolactone, lactide, glycolide lactones, caprolactam and combinations thereof, is initiated by the hydroxyl, amine, carboxylic acid, thiol or any other end group or any other reactive end group present at the A, B, M or N chain, under the influence of a catalyst selected from a group consisting of stannous octanoate or any other catalyst related. The Xₙ-A-Xₙ or Xₙ-B-Xₙ type triblock or the M-Xₙ or N-Xn type diblock or Xₙ-A(Xₙ)ₓ-Xₙ or Xₙ-B(Xₙ)_{y}-Xₙ type multiblock is generated at this point, and its molecular weight is a function of both the molecular weight of the block A, B, M or N, and the length of the polyester, polyamide, poly(anhydride) block(s) or any other related block, preferably PLA, PGA or PCL lateral block(s). In the next step of the synthesis, intermediate segments are formed by reacting the triblock, diblocks or multiblocks with E, preferably phosgene, to obtain E-Xₙ-A-Xₙ-E or E-Xₙ-B-Xₙ-E pentablocks or M-Xₙ-E or N-Xₙ-E triblocks or E-Xₙ-A(Xₙ)_{y}(E)_{y}-Xₙ-E or E-Xₙ-B(Xₙ)_{y}(E)_{y}-Xₙ-E multiblocks. The final polymer is obtained by reacting the pentablock E-Xₙ-A-Xₙ-E or the triblock M-Xₙ-E or the multiblock E-Xₙ-A(Xₙ)_{y}(E)_{y}-Xₙ-E with the segment B, or the pentablock E-Xₙ-B-Xₙ-E or the triblock N-Xₙ-E or multiblock E-Xₙ-B(Xₙ)_{y}(E)_{y}-Xₙ-E, with segment A.

b) Random Polymers

In order to synthesize the Xₙ-A-Xₙ or Xₙ-B-Xₙ triblocks or M-Xₙ or N-Xn diblocks or Xₙ-A(Xₙ)ₓ-Xₙ or Xₙ-B(Xₙ)_{y}-Xₙ multiblocks, segments A, B, M or N, terminated with hydroxy, amine or carboxylic acid groups, or any other group able of opening lactones and lactams, are reacted with the hydroxyacid, the aminoacid, the respective lactone or lactam, anhydride or a related monomer as otherwise described herein, to produce an Xₙ-A-X_{or X}ₙ-B-Xₙ triblock or an M-Xₙ or N-Xn diblock or Xₙ-A(Xₙ)ₓ-Xₙ or Xₙ-B(Xₙ)_{y}-Xₙ multiblocks. Once the triblock or the diblock or the multiblock is formed, it is mixed with the hydrophobic or hydrophilic segment, as fit, and is reacted with the chain extender E in order to obtain a random polymer. The synthesis of polymers with n equal to 0, is carried out eliminating the first step of formation of the Xₙ-A-Xₙ or Xₙ-B-Xₙ triblock or M-Xₙ or N-Xₙ diblock or Xₙ-A(Xₙ)ₓ-Xₙ or Xₙ-B(Xₙ)_{y}-Xₙ multiblocks, and the bifunctional or multifunctional segment A or B or the monofunctional segment M or N, respectively, is mixed with the hydrophobic segment B or the hydrophilic segment A, and finally reacted with the chain extender E.

The Brookfield Viscometer was the main analytical tool used to determine the rheological behavior of the different systems, as a function of the temperature. Ti (the temperature at which the viscosity of the system starts climbing), a functional parameter of the utmost importance, as well as η*, the viscosity at physiological temperature, were determined.

While the invention will now be described in connection with certain preferred embodiments in the following examples and with reference to the accompanying figures so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

In the drawings:

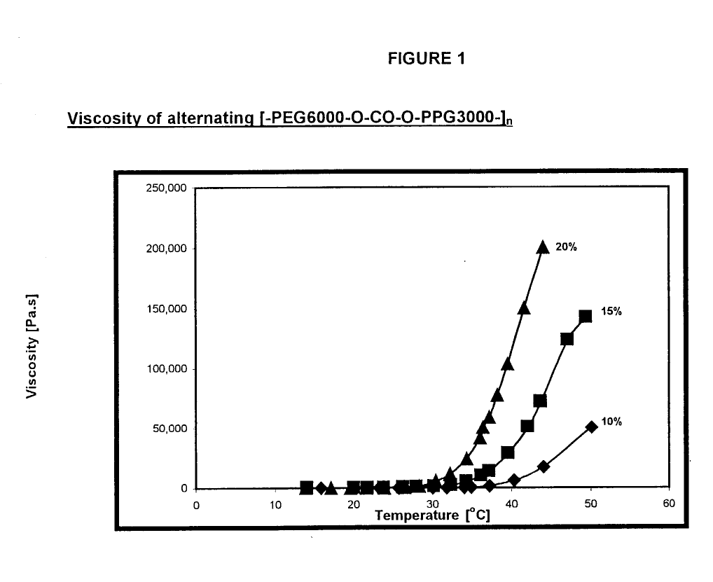

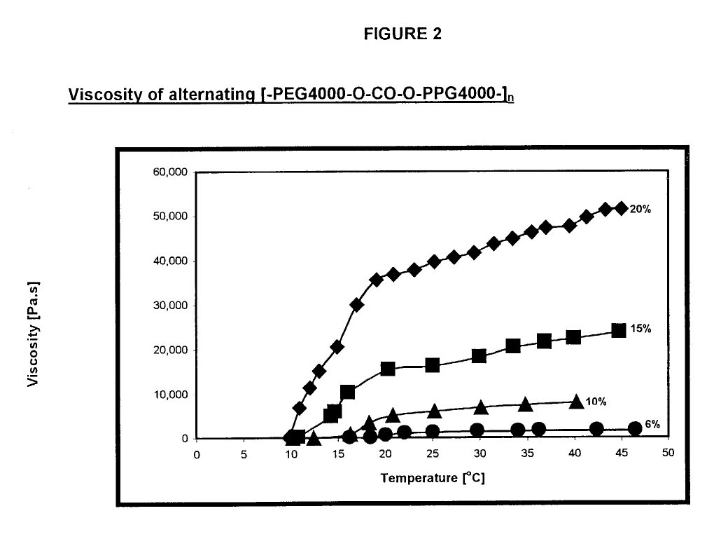

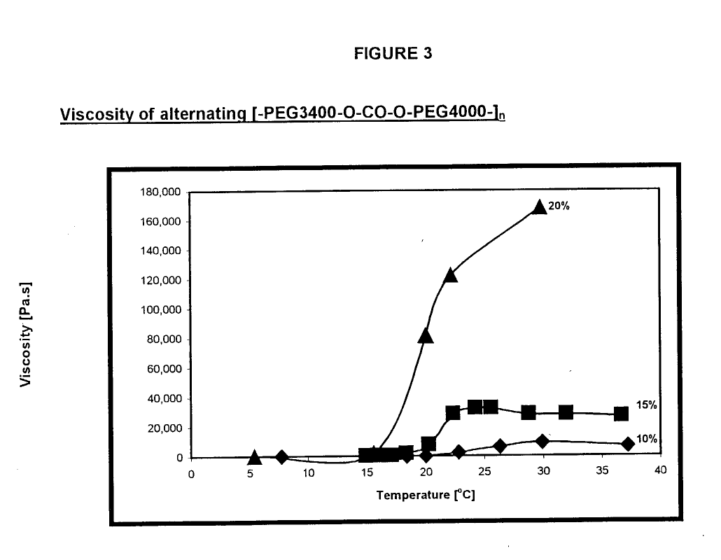

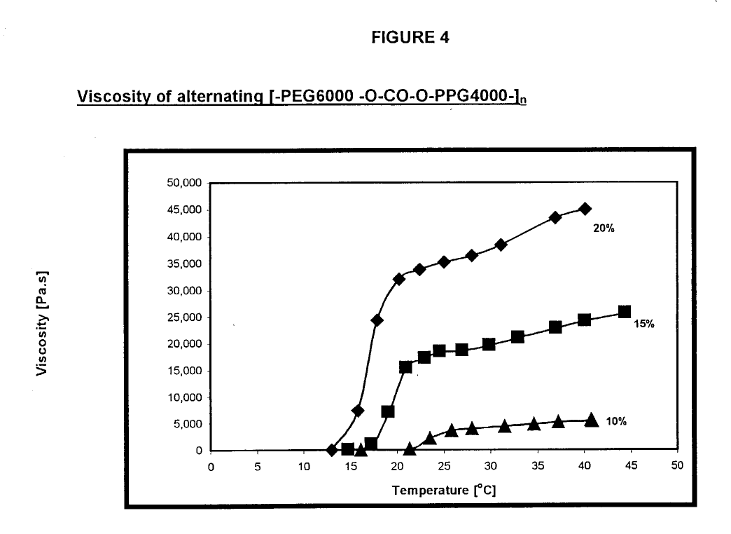

FIGS. 1-4 are graphical representations of viscosities of different polymers;

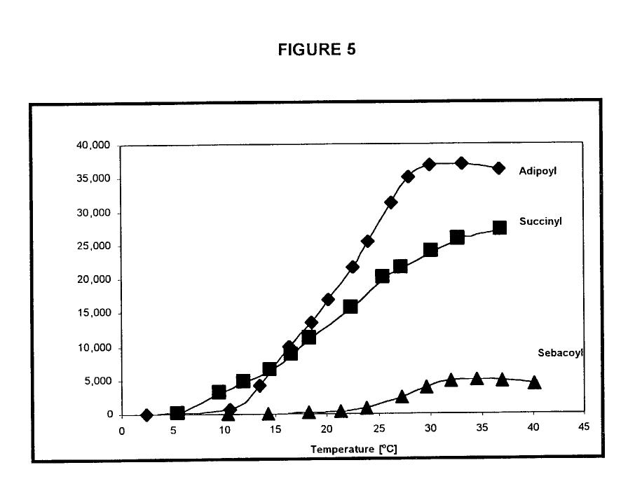

FIG. 5 is a graphical representation of the rheological behavior of adipoyl, succinyl and sebacoyl;

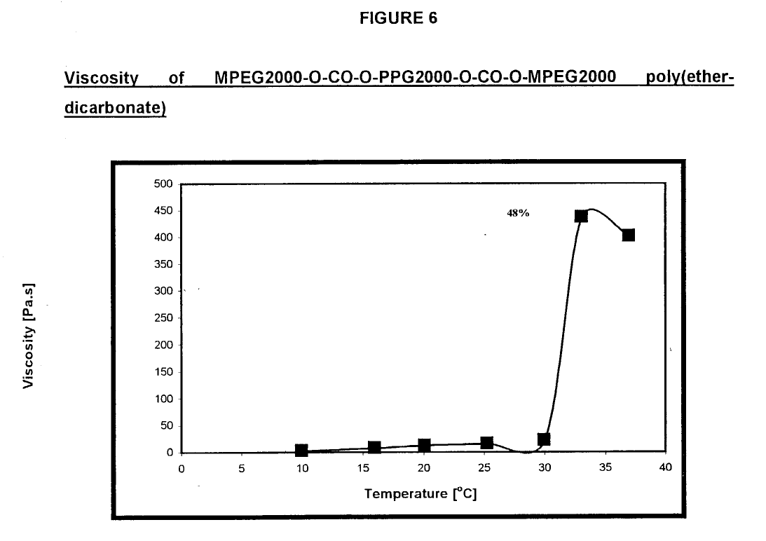

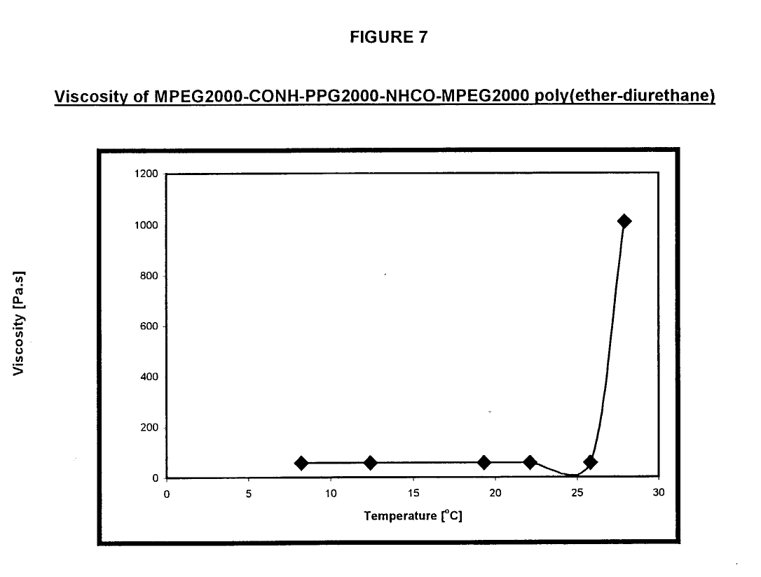

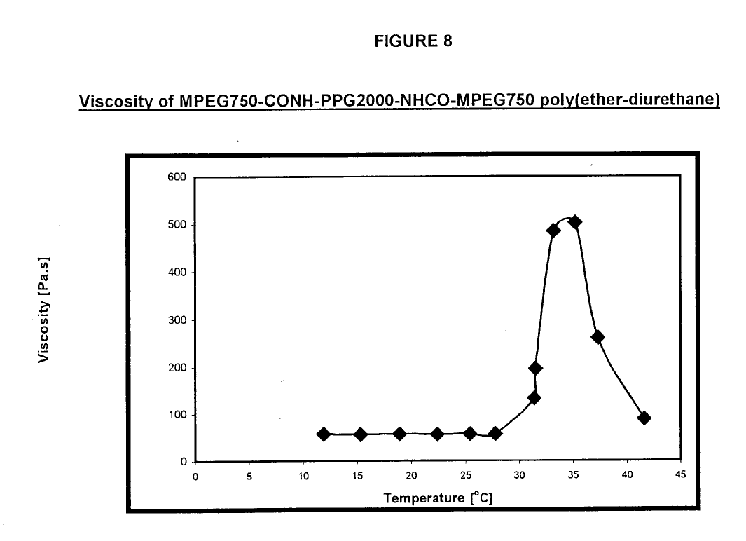

FIGS. 6-8 are graphical representations of the viscosity of water solutions of further polymers; and

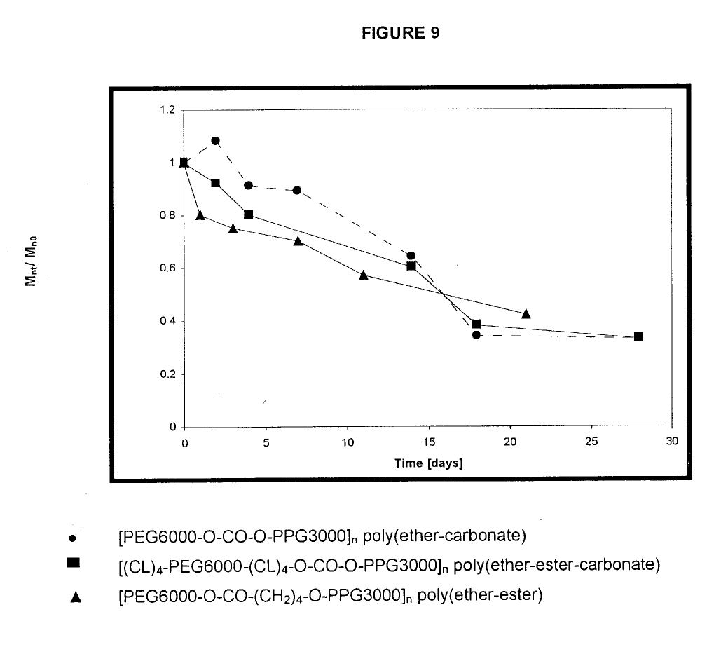

FIG. 9 is a graphical representation of the degradation of poly(ether-carbonate), poly(ether-ester-carbonate) and poly(ether-ester) with time.

### EXAMPLES

The synthesis of the polymers is presented in the following examples. In general, the solvents used are of analytical grade and were dried adding Molecular Sieves 4A of BDH Co., poly(ethylene oxide), poly(ethylene oxide) monomethyl ether, poly(propylene oxide), polytetramethylene glycol, polycaprolactone diol and ε-caprolactone were supplied by Aldrich Co., lactide was purchased from Boehringer Ingelheim Co., the polyoxopropylene diamine was provided by Texaco Co. and the phosgene chloroformic solution was prepared in our laboratory from 1,3,5-trioxane and carbon tetrachloride, using aluminum chloride as catalyst according to to Eisenschtadt et al. (I. N. Eisenschtadt et al., Zhurnal prikladnoi khimii 21 (1968) 1380). Both poly(ethylene oxide) and poly(propylene oxide) were dried under vacuum at 100-110° C. for one hour, before using. The polyoxopropylene diamine (Jeffamine) was treated under vacuum at RT in order to eliminate traces of NH₃ or other volatile amine compounds that could be present.

### Example 1

### Synthesis of alternating [-PEG6000-O—CO—O-PPG3000-]ₙ poly(ether-carbonate)

i) Synthesis of phosgene and preparation of the chloroformic solution

The phosgene was generated by reacting 1,3,5 trioxane (15 g) with carbon tetrachloride (100 g) using aluminum trichloride (30 g) as the catalyst. The phosgene vapors were bubbled in weighed chloroform and the phosgene concentration (w/w) was calculated by weight difference (between 9% and 11%). Due to phosgene's high toxicity, the solution was handled with extreme care and all the work was conducted under a suitable hood.

ii) Synthesis of PEG6000 dichloroformate (ClCO—O-PEG6000-O—COCl)

30.3 grams of dried PEG6000 (molecular weight 6,000) were dissolved in 50 ml dried chloroform in a 250 ml flask. 66 gram of chloroformic solution of phosgene 3% w/w (100% molar excess to PEG) were added to the PEG and the mixture was allowed to react at 60° C. for 4 h with magnetic stirring and a condenser in order to avoid solvent and phosgene evaporation. The reaction flask was connected to a NaOH trap (20% w/w solution in water/ethanol 1.1) in order to trap the phosgene that could be released during the reaction. Once the reaction was completed, the system was allowed to cool down to RT and the excess of phosgene was eliminated by vacuum. The FT-IR analysis showed the characteristic peak at 1777 cm⁻¹ belonging to the chloroformate group vibration.

iii) Synthesis of alternating [-PEG6000-O—CO—O-PPG3000-]ₙ poly(ether-carbonate)

15.2 ms of dried PPG3000 (molecular weight 3,000) were added to ClCO -PEG6000-COCl produced in a) at RT. The mixture was cooled to 5° C. in an ice bath and 6.3 grams pyridine dissolved in 20 ml chloroform were added dropwise over a 15 min period. Then, the temperature was allowed to heat up to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained. The material displayed a melting endotherm at 53.5° C. and the FT-IR analysis showed the characteristic carbonate group peak at 1746 cm⁻¹. The molecular weight of the polymer produced was Mₙ 36,400 (M_{w}/Mₙ=1.28), as determined by GPC. The PEG/PPG block ratio in the final product was determined by ¹H-NMR using a calibration curve obtained from different blends having various PEG6000/PPG3000 ratios and was 1.78, whereas the PEO/PPO ratio was 4.7.

### Example 2

### Synthesis of alternating [-PEG4000-O—CO—O-PPG4000-]ₙ poly(ether-carbonate)

i) Synthesis of phosgene and preparation of the chloroformic solution

The synthesis of phosgene and preparation of the chloroformic solution were described in Example 1i).

ii) Synthesis of PEG4000 dichloroformate (ClCO—O-PEG4000-O—COCl)

The procedure described in example 1ii) was essentially repeated, except that 20.2 grams (0.005 mol) PEG4000 (molecular weight 4,000) and 20 grams of the chloroformic solution of phosgene 7.7% w/w (100% molar excess to PEG), were used. The FT-IR analysis showed the characteristic peak at 1777 cm⁻¹ belonging to the chloroformate group vibration.

iii) Synthesis of alternating [-PEG4000-O—CO—O-PPG4000-]ₙ poly(ether-carbonate)

The procedure in example 1iii) was essentially repeated, except that 20.1 grams (0.005 mol) PEG4000 (molecular weight 4,000) and 7.9 grams pyridine were used. A light yellow powder was obtained. The product showed T_{g} at −74° C. and Tₘ at 50° C. and FT-IR analysis showed the characteristic carbonate peak at 1746 cm⁻¹. The molecular weight of the polymer produced was Mₙ 25,500 (M_{w}/Mₙ=1.53), as determined by GPC. The PEG/PPG block ratio, as determined by ¹H-NMR, was 1.27, whereas the molar ratio PEO/PPO was 1.67.

### Example 3

### Synthesis of alternating [-PEG3400-O—CO—O-PPG4000-]ₙ poly(ether-carbonate)

i) Synthesis of phosgene and preparation of the chloroformic solution

The synthesis of phosgene and preparation of the chloroformic solution were described in Example 1i).

ii) Synthesis of PEG3400 dichloroformate (ClCO—O-PEG3400-O—COCl)

The procedure in example 1ii) was essentially repeated, except that 20 grams (0.0059 mol) PEG3400 (molecular weight 3,400) and 19.5 grams of the chloroformic solution of phosgene 5.9% w/w (100% molar excess to PEG), were used. The FT-IR analysis showed the characteristic peak at 1777 cm⁻¹ belonging to the chloroformate group vibration.

iii) Synthesis of alternating [-PEG3400-O—CO—O-PEG4000-]ₙ poly(ether-carbonate)

The procedure in example 1iii) was essentially repeated, except that 23.5 grams (0.0059 mol) PPG4000 (molecular weight 4,000) and 7.9 grams pyridine were used. The product was a light yellow powder, which showed a T_{g} at −73° C. and Tₘ at 45° C., and FT-IR analysis showed the carbonate characteristic peak at 1746 cm⁻¹. The molecular weight of the polymer produced was Mₙ 29,200 (M_{w}/Mₙ=1.35), as determined by GPC. The PEG/PPG block ratio determined by ¹H-NMR using a calibration curve obtained from different ratio PEG3400/PPG4000 blends and was. The polymer produced presented Mₙ 12,500 (M_{w}/Mₙ=2.38). The PEG/PPG block molar ratio determined by ¹H-NMR using a calibration curve obtained from different ratio PEG4000/PPG4000 blends and was 1.15, whereas the molar ratio PEO/PPO was 1.3.

### Example 4

### Synthesis of alternating [-PEG6000-O—CO—O-PPG4000-]ₙ poly(ether-carbonate)

i) Synthesis of phosgene and preparation of the chloroformic solution

The synthesis of phosgene and preparation of the chloroformic solution were described in Example 1i).

ii) Synthesis of PEG6000 dichloroformate (ClCO—O-PEG6000-O—COCl)

The synthesis of PEG6000 dichloroformate was described in Example 1ii).

iii) Synthesis of alternating [-PEG6000-O—CO—O-PEG4000-]ₙ poly(ether-carbonate)

The procedure in example 1iii) was essentially repeated, except that 20.3 grams (0.0051 mol) PPG4000 (molecular weight 4,000) and 7.9 grams pyridine were used. The product was a light yellow powder, which showed a T_{g} at −73° C. and Tₘ at 45° C., and FT-IR analysis showed the carbonate characteristic peak at 1746 cm⁻¹. The molecular weight of the polymer produced was Mₙ 29,200 (M_{w}/Mₙ=1.35), as determined by GPC. The PEG/PPG block ratio determined by ¹H-NMR using a calibration curve obtained from different ratio PEG3400/PPG4000 blends and was. The polymer produced presented Mₙ 12,500 (M_{w}/Mₙ=2.38). The PEG/PPG block molar ratio determined by ¹H-NMR using a calibration curve obtained from different ratio PEG4000/PPG4000 blends and was 1.15, whereas the molar ratio PEO/PPO was 1.3.

### Example 5

### Synthesis of alternating [(Caprolactone)₄-PEG6000-(Caprolactone)₄-O—CO—O-PPG3000]ₙ poly(ether-ester-carbonate)

i) Synthesis of (Caprolactone)₄-PEG6000-(Caprolactone)₄ triblock

30.3 g of PEG6000 were dried at 120° C. under vacuum for 2 hours. Then, 10.1 g caprolactone and 0.05 g stannous 2-ethyl-hexanoate were added. The reaction mixture was heated at 145° C. for 2.5 hours in a dry nitrogen atmosphere. Finally, the reaction mixture was cooled to RT, dissolved in chloroform, precipitated in petroleum ether and dried at RT.

ii) Synthesis of alternating [(Caprolactone)₄-PEG6000-(Caprolactone)₄-OCO-PPG3000]ₙ poly(ether-ester-carbonate)

a) Synthesis of ClCO-(Caprolactone)₄-PEG6000-(Caprolactone)₄—COCl

40.1 grams of dry (Caprolactone)₄-PEG6000-(Caprolactone)₄ were dissolved in 50 ml dry chloroform in a 250 ml flask. 66 gram of a 3% w/w chloroformic solution of phosgene (100% molar excess to PEG) were added to the PEG and the mixture was allowed to react at 60° C. for 4 h, with magnetic stirring and a condenser in order to avoid solvent and phosgene evaporation. The reaction flask was connected to a NaOH trap (20% w/w solution in water/ethanol 1.1) in order to trap the phosgene that could be released during the reaction. Once the reaction was completed, the system was allowed to cool down to room temperature (RT) and the excess of phosgene was eliminated by vacuum. The FT-IR analysis showed the characteristic absorption band at 1777 cm⁻¹, belonging to the chloroformate group vibration.

b) Synthesis of [(Caprolactone)₄-PEG6000-(Caprolactone)₄-OCO-PPG3000]ₙ polymer

15.2 grams of dry PPG3000 (molecular weight 3,000) were added to ClCO-(Caprolactone)₄-PEG6000-(Caprolactone)₄—COCl produced in i), at RT. The mixture was cooled to 5° C. in an ice bath and 6.3 grams pyridine dissolved in 20 ml chloroform, were added dropwise over a 15 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 6

### Synthesis of alternating [(Caprolactone)₂-PEG4000-(Caprolactone)₂-O—CO—O-PPG4000]ₙ poly(ether-ester-carbonate)

i) Synthesis of (Caprolactone)₂-PEG4000-(Caprolactone)₂ triblock

The procedure in example 5i) was essentially repeated, except that 20.2 g (0.005 mol) PEG4000, 2.8 g (0.012 mol) e-caprolactone and 0.05 g (0.0001 mol) stannous octanoate were used.

ii) Synthesis of alternating poly(ether-ester-carbonate) [(Caprolactone)₄-PEG6000-(Caprolactone)₄-OCO-PPG3000]ₙ

a) Synthesis of ClCO-(Caprolactone)₂-PEG4000-(Caprolactone)₂—COCl

22.8 grams of dry (Caprolactone)₂-PEG4000-(Caprolactone)₂ were dissolved in 50 ml dry chloroform in a 250 ml flask. 66 gram of a 3% w/w chloroformic solution of phosgene (100% molar excess to the triblock) were added to the PEG and the mixture was allowed to react at 60° C. for 4 h, with magnetic stirring and a condenser in order to avoid solvent and phosgene evaporation. The reaction flask was connected to a NaOH trap (20% w/w solution in water/ethanol 1:1) in order to trap the phosgene that could be released during the reaction. Once the reaction was completed, the system was allowed to cool down to room temperature (RT) and the excess of phosgene was eliminated by vacuum. The FT-IR analysis showed the characteristic absorption band at 1777 cm⁻¹, belonging to the chloroformate group vibration.

b) Synthesis of [(Caprolactone)₂-PEG4000-(Caprolactone)₂-OCO-PPG4000]ₙ polymer

20.3 g (0.005 mol) of dry PPG4000 (molecular weight 4000) were added to ClCO-(Caprolactone)₂-PEG4000-(Caprolactone)₂-COCl produced in 6i), at RT. The mixture was cooled to 5° C. in an ice bath and 6.3 grams pyridine dissolved in 20 ml chloroform, were added dropwise over a 15 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 7

### Synthesis of alternating [-PEG6000-O—CO—O-PTMG2900-]ₙ poly(ether-carbonate)

i) Synthesis of phosgene and preparation of the chloroformic solution

The synthesis of phosgene and preparation of the chloroformic solution were described in Example 1i).

ii) Synthesis of PEG6000 dichloroformate (ClCO—O-PEG6000-O—COCl)

The synthesis of PEG6000 dichloroformate was described in Example 1ii).

iii) Synthesis of alternating [-PEG6000-O—CO—O-PTMG2900-]ₙ poly(ether-carbonate)

14.9 g (0.005 mol) of dry PTMG2900 (molecular weight 2900) were dissolved in 20 ml of dry chloroform and added to ClCO-PEG6000-COCl produced in 7i), at RT. The mixture was cooled to 5° C. in an ice bath and 6.6 grams pyridine dissolved in 20 ml chloroform, were added dropwise over a 25 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 8

### Synthesis of alternating [-PEG6000-O—CO—PTMG1000-]ₙ poly(ether-carbonate)

i) Synthesis of phosgene and preparation of the chloroformic solution

The synthesis of phosgene and preparation of the chloroformic solution were described in Example 1i).

ii) Synthesis of PEG6000 dichloroformate (ClCO—O-PEG6000-O—COCl)

The synthesis of PEG6000 dichloroformate was described in Example 1ii).

iii) Synthesis of alternating [-PEG60000-CO—O-PTMG1000-]ₙ poly(ether-carbonate)

5.1 g (0.005 mol) of dry PTMG1000 (molecular weight 4000) were dissolved in 10 ml of dry chloroform and added to ClCO-PEG6000-COCl produced in 8i), at RT. The mixture was cooled to 5° C. in an ice bath and 6.2 grams pyridine dissolved in 20 ml chloroform, were added dropwise over a 30 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 9

### Synthesis of alternating [-PEG6000-O—CO—O-PCL1250-]ₙ poly(ether-ester-carbonate)

i) Synthesis of phosgene and preparation of the chloroformic solution

The synthesis of phosgene and preparation of the chloroformic solution were described in Example 1i).

ii) Synthesis of PEG6000 dichloroformate (ClCO—O-PEG6000-O—COCl)

The synthesis of PEG6000 dichloroformate was described in Example 1ii).

iii) Synthesis of alternating [-PEG6000-O—CO—O-PCL1250-]ₙ poly(ether-carbonate)

6.3 g (0.005 mol) of PCL1250 (molecular weight 1250) were dissolved in 20 ml of dry chloroform and added to ClCO-PEG6000-COCl produced in 9i), at RT. The mixture was cooled to 5° C. in an ice bath and 6.3 grams pyridine dissolved in 20 ml chloroform, were added dropwise over a 19 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 10

### Synthesis of alternating [-PEG6000-O—CO—O-PCL2000-]ₙ poly(ether-ester-carbonate)

i) Synthesis of phosgene and preparation of the chloroformic solution

The synthesis of phosgene and preparation of the chloroformic solution were described in Example 1i).

ii) Synthesis of PEG6000 dichloroformate (ClCO—O-PEG6000-O—COCl)

The synthesis of PEG6000 dichloroformate was described in Example 1ii).

iii) Synthesis of alternating [-PEG6000-O—CO—O-PCL1250-]ₙ poly(ether-carbonate)

10.1 g (0.005 mol) of PCL2000 (molecular weight 2000) were dissolved in 20 ml of dry chloroform and added to ClCO-PEG6000-COCl produced in 9i), at RT. The mixture was cooled to 5° C. in an ice bath and 6.3 grams pyridine dissolved in 20 ml chloroform, were added dropwise over a 22 min period. Then, the temperature was allowed to rise to RT and the reaction was continued for additional 45 minutes. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 11

### Synthesis of random [-PEG6000-O—CO—O-PPG3000-]ₙ poly(ether-carbonate)

i) Synthesis of phosgene and preparation of the chloroformic solution

The synthesis of phosgene and preparation of the chloroformic solution were described in Example 1i).

ii) Synthesis of random [-PEG6000-O—CO—O-PPG3000-]ₙ poly(ether-carbonate)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 22 g of phosgene solution 9% were added dropwise over a period of 1 h 15 min. at RT under magnetic stirring. The reaction was continued for additional 45 minutes at RT. After that, the temperature was risen to 35° C. and the reaction was continued for one additional hour. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 12

### Synthesis of random [-PEG6000-O—CO—CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 1.5 g oxalyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a yellow, brittle and water soluble powder was obtained.

### Example 13

### Synthesis of random [-PEG6000-O—CO—(CH₂)₂CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 1.9 g succinyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried. A brown, brittle and water soluble powder was obtained.

### Example 14

### Synthesis of random [-PEG6000-O—CO—(CH₂)₃—CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 2.1 g glutaryl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and an orange, brittle and water soluble powder was obtained.

### Example 15

### Synthesis of random [-PEG6000-O—CO—(CH₂)₄-CO—O-PPG3000-]ₙ poly(ether-ester)

15.3 grams (0.003 mol) of dry PEG6000 (molecular weight 6,000) and 7.4 g (0.003 mol) of PPG3000 were dissolved in 30 ml dry chloroform in a 250 ml flask. 3.2 g pyridine were added to the reaction mixture. Then 2.2 g adipoyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 16

### Synthesis of random [-PEG6000-O—CO—(CH₂)₈-CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 2.9 g sebacoyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 17

### Synthesis of random [-PEG6000-O—CO-para-Ph-CO—O-PPG3000]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 2.4 g terphtaloyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 18

### Synthesis of random [-PEG6000-O—CO-metha-Ph-CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 2.4 g isophtaloyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 19

### Synthesis of random [-PEG6000-O—CO-ortho-Ph-CO—O-PPG3000-]ₙ poly(ether-ester)

15.1 grams (0.003 mol) of dry PEG6000 (molecular weight 6,000) and 7.6 g (0.003 mol) of PPG3000 were dissolved in 30 ml dry chloroform in a 250 ml flask. 3.2 g pyridine were added to the reaction mixture. Then 2.4 g phtaloyl chloride in 20 ml of dry chloroform were added dropwise over a period of 30 min. at 40° C. under magnetic stirring. After that, the temperature was risen to 60° C. and the reaction was continued for one additional hour and half. The polymer produced was separated from the reaction mixture by adding it to about 600 ml petroleum ether 40-60. The lower phase of the two-phase system produced was separated and dried at RT. Finally, the polymer was washed with portions of petroleum ether and dried, and a light yellow, brittle and water soluble powder was obtained.

### Example 20

### Synthesis of MPEG2000-CONH-PPG2000-NHCO-MPEG2000 poly(ether-diurethane)

i) Synthesis of phosgene and preparation of the chloroformic solution

The synthesis of phosgene and preparation of the chloroformic solution were described in Example 1i).

ii) Synthesis of MPEG2000 chloroformate (MPEG2000-COCl)

The procedure in example 1i) was substantially repeated, except that 10.5 grams (0.005 mol) MPEG2000 (molecular weight 2,000) and 35 grams chloroformic solution of phosgene 3% w/w (100% molar excess to MPEG) were used. The FT-IR analysis was fit for the chloroformate group vibration.

iii) Synthesis of MPEG2000-CONH-PPG2000-NHCO-MPEG2000 polymer

The procedure in example 1ii) was substantially repeated, except that 5 grams (0.0025 mol) Jeffamine D-2000 (molecular weight 2,000) and 1.6 grams pyridine were used. The product was a slight yellow solid at RT. The product shows T_{g} at −69° C. and Tₘ at 50° C. and FT-IR analysis showed characteristic peak to the urethane group at 1736 cm⁻¹. The polymer produced presented Mₙ 8,900 (M_{w}/Mₙ=1.27).

### Example 21

### Synthesis of MPEG750-CONH-PPG2000-NHCO-MPEG750 poly(ether-diurethane)

i) Synthesis of phosgene and preparation of the chloroformic solution

The synthesis of phosgene and preparation of the chloroformic solution were described in Example 1i).

ii) Synthesis of MPEG750 chloroformate (MPEG750-COCl)

The procedure in example 1i) was substantially repeated, except that 10 grams (0.013 mol) MPEG750 (molecular weight 750) and 66 grams chloroformic solution of phosgene 3% w/w (50% molar excess to MPEG) were used. The FT-IR analysis was fit for the chloroformate group vibration.

iii) Synthesis of MPEG750-CONH-PPG2000-NHCO-MPEG750 polymer

The procedure in example 1ii) was substantially repeated, except that 14 grams (0.007 mol) Jeffamine D-2000 (molecular weight 2,000) and 4.27 grams pyridine were used. The material was a dark yellow waxy solid at RT. The product shows T_{g} at −71° C. and Tₘ at 24° C. and FT-IR analysis showed characteristic peak to the urethane group at 1720 cm⁻¹. The polymer produced presented Mₙ 2,320 (M_{w}/Mₙ=1.28).

### Example 22

### Synthesis of MPEG2000-O—CO—O-PPG2000-O—CO—O-MPEG2000 poly(ether-dicarbonate)

i) Synthesis of phosgene and preparation of the chloroformic solution

The synthesis of phosgene and preparation of the chloroformic solution were described in

### Example 1i).

ii) Synthesis of MPEG2000 chloroformate (MPEG2000-COCl)

The procedure of the MPEG2000 chloroformate synthesis was described in example 20i).

iii) Synthesis of MPEG20000-CONH-PPG2000-NHCO-MPEG2000 polymer

The procedure in example 20ii) was substantially repeated, except that 5 grams (0.0025 mol) PPG2000 (molecular weight 2,000) and 1.6 grams pyridine were used. The product was a slight yellow solid at RT. The product shows T_{g} at −71° C. and Tₘ at 51° C. and FT-IR analysis showed characteristic peak to the carbonate group at 1720 cm⁻¹. The polymer produced presented M_{w}/Mₙ=1.27).

### Example 23

a) Viscosity of Aqueous Solution of Polymers

The viscosity of water solutions of the different polymers was determined in a Brookfield Viscometer DV-II+ with temperature control and different spindles as required at 0.05 RPM and the graphical representations thereof are shown in FIGS. 1-4.

b) Viscosity of random [-PEG6000-O—CO—O-PPG3000-]ₙ

The modification of the PEG6000/PPG3000 in the synthesis step rendered different PEO/PPO ratios in the final poly(ether-carbonate). The following table exemplifies the different PEO/PPO ratios achieved as well as the rheological parameters in 15% aqueous solutions. Where Ti is the gelation temperature.

| PEG [wt %] | Tᵢ [° C.] | η₃₇° C. [Pa.s] |
|---|---|---|
| 81 | 27 | 17,000 |
| 77 | 21 | 62,000 |
| 71 | 14 | 83,400 |
| 62 | 10 | 42,000 |

c) Viscosity of random aliphatic poly(ether-ester)s

The rheological behavior of three different aliphatic poly(ether-ester)s developed in this invention in 15% w/w aqueous solution and based on adipoyl, succinyl and sebacoyl chain extenders, respectively and the graphical representation thereof, is shown in FIG. 5.

d) Viscosity of Further Aqueous Solutions of Polymers

The viscosity of water solutions of further polymers are set forth in graphical representation in FIGS. 6-8.

### Example 24

### Degradation of poly(ether-carbonate), poly(ether-ester-carbonate) and poly(ether-ester) at 37° C.

The molecular weight decrease with time (Mₙₜ) with time related to the initial molecular weight (Mₙ₀) is shown in graphical representation in FIG. 9.

It must to be understood that the examples and embodiments described hereinabove are for the purposes of providing a description of the present invention by way of example and are not to be viewed as limiting the present invention in any way. Various modifications or changes that may be maded to that described hereinabove by those of ordinary skill in the art are also contemplated by the present invention and are to be included within the spirit and purview of this application.

What is claimed is:

1) A responsive polymeric system, comprising:
a polymeric responsive component capable of undergoing a transition that results in a sharp increase in viscosity in response to a change in temperature at a predetermined body site;
wherein the polymeric component comprises hydrophilic and hydrophobic segments covalently bound within said polymer component, by at least one chain extender or coupling agent, having at least 2 functional groups;
wherein the hydrophilic and hydrophobic segments do not display Reverse Thermal Gelation behavior of their own at clinically relevant temperatures and;
wherein the viscosity of said polymeric component increases by at least about 2 times upon exposure to a predetermined trigger.

2. A responsive polymeric system, according to claim 1, wherein said responsive polymeric component has a formula selected from a group consisting of:
a) [-Xₙ-A-Xₙ-E-B-E-]ₘ
b) [-Xₙ-B-Xₙ-E-A-E-]ₘ,
c) M-Xₙ-E-B-E-Xₙ-M
d) N-Xₙ-E-A-E-Xₙ-N
e) [-E-Xₙ-A(Xₙ)_{y}(E)_{y}(B)_{y}-Xₙ-E-B-]ₘ and
f) [-E-Xₙ-B(Xₙ)_{y}(E)_{y}(A)_{y}-Xₙ-E-A-]ₘ;
wherein segments A are bifunctional, trifunctional or multifunctional hydrophilic segments and M are monofunctional hydrophilic segments, respectively;
wherein segments B are bifunctional, trifunctional or multifunctional hydrophobic segments and N are monofunctional hydrophobic segments, respectively;
wherein segments X are bifunctional degradable segments;
wherein E are bi, tri or multifunctional chain extenders or coupling molecules,
wherein n and m denote the respective degrees of polymerization and y designates the additional functionality of the segment above 2.

3) The responsive polymeric system of claim 1, wherein said predetermined trigger is temperature and said system displays said increase in viscosity upon heating.

4) The responsive polymeric system of claim 3, wherein said polymeric system undergoes a sharp increase in viscosity in response to a change in temperature from a lower temperature to body temperature.

5) The responsive polymeric system of claim 1, wherein said responsive polymeric component is biodegradable.

6) The responsive polymeric system of claim 1, wherein said hydrophilic segment A is selected from a hydrophilic bifunctional segment selected from the group consisting of —OH, —SH, —COOH, —NH₂, —CN and a —NCO terminated poly(oxoethylene), and a trifunctional segment selected from a group consisting of poly(oxoethylene triol), poly(oxoethylene triamine), poly(oxoethylene tricarboxylic acid), and ethoxylated trimethylolpropane.

7) The responsive polymeric system of claim 1, wherein said monofunctional hydrophilic segment M is a hydrophilic monofunctional segment, selected from a group consisting of —OH, —SH, —COOH, —NH₂, —CN and a —NCO-terminated poly(oxoethylene) monomethylether.

8) The responsive polymeric system of claim 1, wherein said hydrophobic segment B is selected from a hydrophobic bifunctional component selected from a group consisting of a —OH, —SH, —COOH, —NH₂, —CN and a —NCO terminated polyoxyalkylene polymer, polytetramethylene glycol (PTMG)), polyesters, polyamides and polyanhydrides, or a trifunctional segment selected from the group consisting of poly(oxopropylene triol), poly(oxopropylene triamine), poly(oxopropylene triacarboxylic acid).

9) The responsive polymeric system according to claim 8 wherein said polyoxyalkylene polymer is selected from the group consisting of poly(propylene glycol) (PPG) and polyoxopropylene diamine.

10) The responsive polymeric system according to claim 8 wherein said polyester is selected from a group consisting of poly(caprolactone), poly(lactic acid), poly(glycolic acid) and copolymers thereof,

11) The responsive polymeric system of claim 1, wherein said monofunctional hydrophobic segment N is an hydrophobic monofunctional segment, selected from the group consisting of —OH, —SH, —COOH, —NH₂, —CN and a —NCO-terminated poly(oxopropylene) monomethylether.

12) The responsive polymeric system of claim 1, wherein said bifunctional chain extender or coupling molecule E is a bifunctional reactive molecule selected from a group consisting of phosgene, aliphatic or aromatic dicarboxylic acids, a reactive derivative (selected from a group consisting of oxalyl chloride, malonyl chloride, succinyl chloride, glutaryl chloride, fumaryl chloride, adipoyl chloride, suberoyl chloride, pimeloyl chloride, sebacoyl chloride, terephtaloyl chloride, isophtaloyl chloride, phtaloyl chloride and/or mixtures thereof or other dicarboxylic acid derivative), aminoacids selected from a group consisting of glycine, alanine, valine, phenylalanine, leucine, isoleucine, oligopeptides selected from a group consisting of Arginine, Glycine, Asparate (RGD) Arginine, Glycine, Asparate, Serine (RGD-S), Arginine, Glutamate, Aspartate, Valine (REDV) aliphatic or aromatic diamines selected from a group consisting of ethylene diamine, propylene diamine and butylene diamine, aliphatic or aromatic diols selected from a group consisting of ethylene diol, propanediol, butylenediol or any other diol, aliphatic or aromatic diisocyanates selected from a group consisting of hexamethylene diisocyanate, methylene bisphenyldiisocyanate, methylene biscyclohexanediisocyanate, tolylene diisocyanate or isophorone diisocyanate, or trifunctional reactive molecules selected from a group consisting of cyanuric chloride, triisocyanates, triamines, triols, aminoacids selected from a group consisting of lysine, serine, threonine, methionine, asparagine, glutamate, glutamine, histidine, aminoacid having three functional groups, oligopeptides or any other trifunctional reactive molecule, having the appropriate terminal groups or multifunctional coupling molecule.

13) The responsive system of claim 12, wherein E is selected from the group consisting of phosgene, diisocyantes, aminoacids, oligopeptides and bifunctional carboxylic acid derivatives, and combinations thereof.

14) The responsive system of claim 12, wherein E comprises combinations of the functional groups defined therein, said combinations forming reaction products selected from the group consisting of poly(ether-carbonate)s, poly(ether-ester)s, poly(ether-urethane)s, derivatives of chlorotriazine, polyimides, polyureas and combinations thereof.

15) The responsive system of claim 12, wherein E comprises poly(ether-carbonate)s, poly(ether-ester)s, poly(ether-urethanes),

16) The responsive system of claim 1, wherein said biodegradable segment X, is selected from a group consisting of esters, amides, carbonates and anhydride groups and combinations thereof.

17) The responsive system of claim 1, wherein said biodegradable segment X, is selected from a group consisting of aliphatic esters or oligo or polyesters, aminoacids, oligo or polypeptides, saccharides and polysaccharides,

18) The responsive polymeric system of claim 1, in combination with a molecule to be delivered into the body.

19) The responsive polymeric system of claim 18, wherein said molecule displays biological activity.

20) The responsive polymeric system of claim 1, comprising at least one molecule displaying biological activity, to be delivered into the body, selected from a group consisting of drugs, enzymes, hormones, growth factors, proteins, olipeptides, and angiogenic factors.

21) The responsive polymeric system of claim 1, wherein said responsive system contains materials of biological source.

22) The responsive polymeric system of claim 1, wherein said responsive system contains living cells.

23) The responsive polymeric system of claim 1, wherein said responsive system contains inorganic components of biological origin.

24) The responsive polymeric system of claim 1, wherein said responsive system contains inorganic components of biological origin selected from a group consisting of tricalcium phosphate, hydroxyapatite and combinations thereof.

25) The responsive polymeric system of claim 1, wherein said responsive system contains components of biological origin selected from a group consisting of elastin, collagenous material, albumin, fibrinous material, demineralized tissue, a cellular tissue matrix and combinations thereof.

26) An injectable system comprising an aqueous based solvent and a responsive polymeric system according to claim 1.
